(19) United States
(12) Patent Application Publication
(10) Pub. No.: US 2023/0313277 A1
(43) Pub. Date: Oct. 5, 2023

(54) QUENCHER AND USE THEREOF

(71) Applicant: SFC CO., LTD., Cheongju-si (KR)

(72) Inventors: Do Min LEE, Cheongju-si (KR); Ho Young SI, Cheongju-si (KR); Jae Hyung JO, Cheongju-si (KR); Goutam MASANTA, Cheongju-si (KR); Ju Man SONG, Cheongju-si (KR); Min Su EUM, Cheongju-si (KR); Jong Tae JE, Cheongju-si (KR)

(73) Assignee: SFC CO., LTD., Cheongju-si (KR)

(21) Appl. No.: 18/332,137

(22) Filed: Jun. 9, 2023

Related U.S. Application Data
(63) Continuation of application No. PCT/KR2021/017728, filed on Nov. 29, 2021.

(30) Foreign Application Priority Data
Dec. 9, 2020 (KR) 10-2020-0171414
Nov. 25, 2021 (KR) 10-2021-0164046

(51) Int. Cl. C12Q 1/6818; C09B 11/24; C09K 11/06; C12Q 1/6844

(52) U.S. Cl. CPC C12Q 1/6818; C09B 11/24; C09K 11/06; C12Q 1/6844

(57) ABSTRACT

The present invention relates to a quencher which exhibits a quenching effect on a fluorescent material exhibiting luminescence at an excited energy level, and various uses thereof.

### CROSS-REFERENCE TO RELATED APPLICATION

This application is based on the PCT Application No. PCT/KR2021/017728, filed on Nov. 29, 2021, and claims the benefit of priority from Korean Patent Application No. 10-2021-0164046, filed on Nov. 25, 2021 and Korean Patent Application No. 10-2020-0171414, filed on Dec. 9, 2020, the disclosures of which are incorporated herein by reference in its entirety.

### BACKGROUND

### 1. Field of the Invention

The present invention relates to a quencher having a quenching effect on a fluorescent material exhibiting luminescence at an excited energy level, and various uses thereof.

### 2. Discussion of Related Art

A quencher is a molecule capable of quenching the fluorescence of a fluorescent molecule, and a dye having a property of absorbing light is generally used.

A quenching phenomenon is known to occur through a mechanism including fluorescence resonance energy transfer (FRET), photo-induced electron transfer and dye coagulation such as H-dimer formation.

When a quencher is used to control or quench the fluorescence of a fluorescent dye, it is most important whether the absorption wavelength range of a quenching dye covers (overlaps) a considerable part or the entire wavelength region of fluorescence exhibited by the fluorescent dye.

In order to obtain a quenching effect, the length between the fluorescence dye and the quencher is also important, and for example, the number of bases in the case of DNA or the number of amino acids in the case of a peptide/protein is considered. To obtain a higher quenching effect, the length of a linker labeled with a fluorescence dye and a quencher may be adjusted.

In the case of a quencher generally used commercially in the field of biology, although a fluorescence-fluorescent dye combination, which uses a FRET phenomenon, has been widely used, a dye structure that does not emit light but only absorbs light is generally selected. The combined fluorescent-quenching and fluorescent-fluorescent dyes may impart a kind of on/off function of fluorescence because the original fluorescence is restored or intensified when the distance between the two dyes increases or the two dyes are separated from a biomolecule, and in consideration of such a characteristic, may be widely used in designing biosensors or activation probes that can respond to biomarkers for specific proteins/enzymes.

When fluorescent or quenching dyes used in the field of biology are used alone, they are only limited to FDA-approved dyes such as indocyanine green or methylene blue, and a reactive group capable of binding to a substituent of a biomolecule is introduced. While various types of reactive groups described above are known, substituent selectivity, reaction rate, yield, reproducibility and stability have been verified by researchers since long ago. In recent years, reactive groups introduced to a dye for practical research or commercial purposes are limited to a few.

For example, the most frequently used reactive groups for binding with an amine group of a protein molecule are succinimidyl ester and isothiocyanate, the most frequently used reactive group for binding with a thiol group of a protein molecule is maleimide, and as a reactive group for binding with a hydroxyl group of a protein molecule, dichlorotriazine is mainly used.

However, in most cases, the reactive groups are bound by substitution or it is difficult to maintain a long-term reaction and storage stability under a water-soluble condition.

### SUMMARY OF THE INVENTION

The present invention is directed to providing a novel quencher as a compound that can be widely used to observe the identification of a biomolecule in the field of optical imaging.

The present invention is also directed to providing an oligonucleotide, a composition and a support for detecting a nucleic acid, and a method of detecting a nucleic acid, including the novel quencher.

To solve the above-described technical problems, according to one aspect of the present invention, a quencher represented by Formula 1 or 2 below is provided.

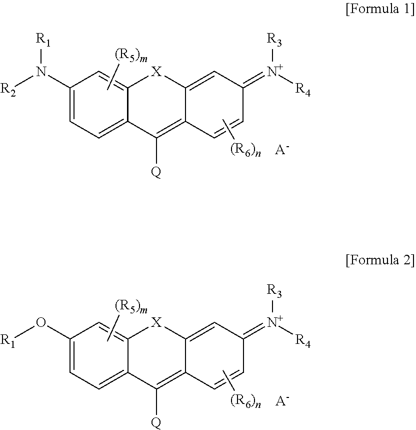

Here, Q is represented by Formula 3 or 4 below,

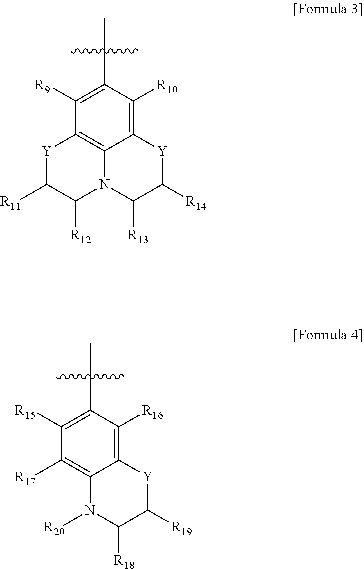

R₁ to R₆ and R₉ to R₂₀ are each independently (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted esters, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide,
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(4) a protecting group selected from an alcohol-derived protecting group, an amide-derived protecting group, a carbonyl-derived protecting group, a carboxyl acid-derived protecting group, a phosphate-derived protecting group, and an alkyne-derived protecting group,
m and n are each independently an integer of 0 to 3,
X is O, S, CR₇R₈, or SiR₇R₈,
Y is O or S,
R₇ and R₈ are each independently selected from substituted or unsubstituted C₁-C₄₀ alkyl, substituted or unsubstituted C₁-C₄₀ heteroalkyl having at least one hetero atom, substituted or unsubstituted C₃-C₃₀ cycloalkyl, substituted or unsubstituted C₃-C₃₀ heterocycloalkyl having at least one hetero atom, substituted or unsubstituted aryl, and substituted or unsubstituted heteroaryl, or form a ring by bonding them together,
at least one of R₁ to R₂₀ is (1) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(2) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(3) a protecting group selected from an alcohol-derived protecting group, an amide-derived protecting group, a carbonyl-derived protecting group, a carboxylic acid-derived protecting group, a phosphate-derived protecting group, and an alkyne-derived protecting group,
R₉, R₁₀, R₁₅ and R₁₆ are not electron-withdrawing groups, and
the case in which all of R₉ to R₁₄ or R₁₅ to R₁₉ are hydrogen is excluded.

In addition, according to another aspect of the present invention, a quencher represented by Formula 5 or 6 below is provided.

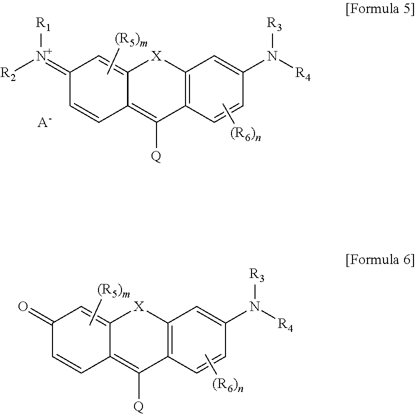

Here, Q is represented by Formula 3 or 4 below,

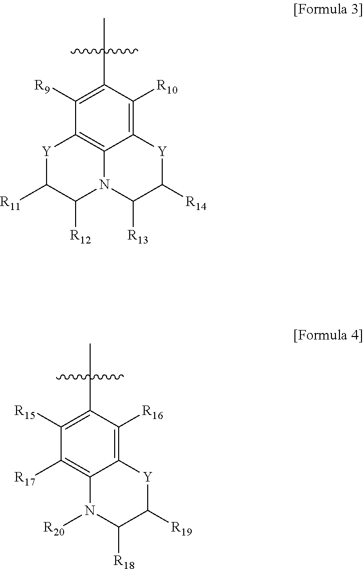

R₁ to R₆ and R₉ to R₂₀ are each independently (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide,
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(4) a protecting group selected from an alcohol-derived protecting group, an amide-derived protecting group, a carbonyl-derived protecting group, a carboxylic acid-derived protecting group, a phosphate-derived protecting group, and an alkyne-derived protecting group, m and n are each independently an integer of 0 to 3,
X is O, S, CR₇R₈, or SiR₇R₈,
Y is O or S,
R₇ and R₈ are each independently selected from substituted or unsubstituted C₁-C₄₀ alkyl, substituted or unsubstituted C₁-C₄₀ heteroalkyl having at least one hetero atom, substituted or unsubstituted C₃-C₃₀ cycloalkyl, substituted or unsubstituted C₃-C₃₀ heterocycloalkyl having at least one hetero atom, substituted or unsubstituted aryl, and substituted or unsubstituted heteroaryl, or form a ring by bonding them together,
at least one of R₁ to R₂₀ is (1) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(2) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(3) a protecting group selected from an alcohol-derived protecting group, an amide-derived protecting group, a carbonyl-derived protecting group, a carboxyl-derived protecting group, a phosphate-derived protecting group, and an alkyne-derived protecting group,
R₉, R₁₀, R₁₅ and R₁₆ are not electron-withdrawing groups, and
the case in which all of R₉ to R₁₀ or R₁₅ to R₁₉ are hydrogen is excluded.

In addition, according to still another aspect of the present invention, an oligonucleotide that includes the above-described quencher, a minor groove binder (MGB), and a fluorophore is provided.

In addition, according to still another aspect of the present invention, a composition for detecting a nucleic acid including the above-described oligonucleotide is provided.

In addition, according to yet another aspect of the present invention, a support for detecting a nucleic acid including the above-described quencher, a support, and a linker connecting the quencher and the support is provided.

In addition, according to yet another aspect of the present invention, a method of detecting a nucleic acid, which includes (a) preparing a reaction mixture including a target nucleic acid, a reagent necessary for amplifying the target nucleic acid, and an oligonucleotide, (b) amplifying the target nucleic acid in the reaction mixture by a chain polymerase reaction, and (c) measuring the fluorescence intensity of the reaction mixture, is provided.

The present invention relates to a quencher exhibiting a quenching effect on a fluorescent material exhibiting a luminescence property at an excited energy level, and various uses thereof, and the quencher according to the present invention can exhibit an excellent quenching property due to high quenching efficiency compared to a conventional quencher.

### BRIEF DESCRIPTION OF THE DRAWINGS

FIG. 1 shows the absorption spectrum of a compound 40 synthesized according to Preparation Example 1 of the present invention.

### DETAILED DESCRIPTION OF EXEMPLARY EMBODIMENTS

To better understand the present invention, certain terms are defined herein for convenience. Unless defined otherwise herein, scientific and technical terms used herein will have meanings commonly understood by those of ordinary skill in the art.

In addition, unless specifically indicated otherwise, terms in a singular form also include plural forms, and terms in a plural form should be understood to include singular forms as well.

Novel Quencher

According to one aspect of the present invention, a quencher represented by Formula 1 or 2 below is provided.

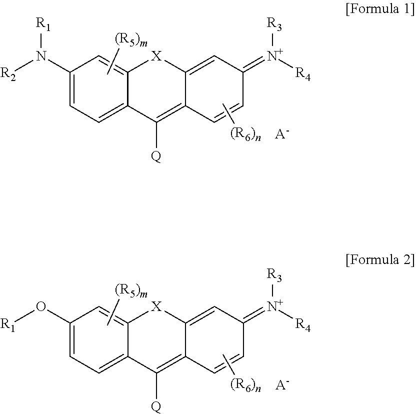

In addition, according to another aspect of the present invention, a quencher represented by Formula 5 or 6 below is provided. Formula 5 represents the resonance structure of Formula 1, and Formula 6 represents the resonance structure of Formula 2. That is, the quencher exists as at least one structure selected from Formula 1 and Formula 5, or exists as at least one structure selected from Formula 2 and Formula 6.

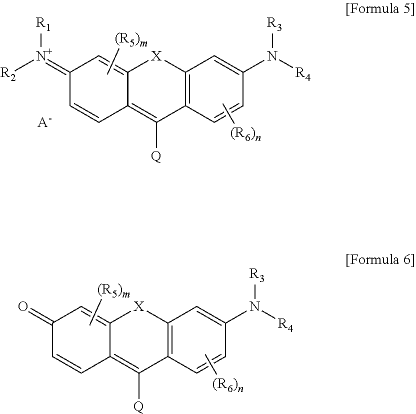

In the quencher represented by Formula 1, 2, 5 or 6, Q is represented by Formula 3 or 4 below.

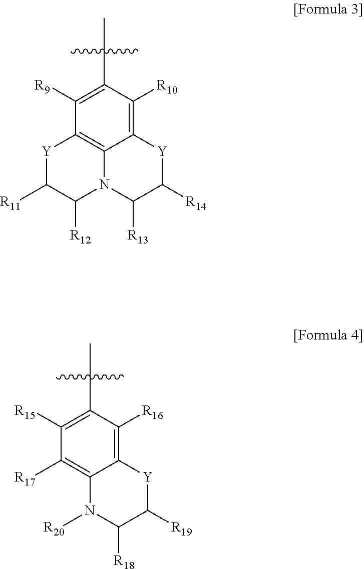

In the quencher represented by Formula 1, 2, 5 or 6, R₁ to R₆ and R₉ to R₂₀ are each independently (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂P cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide,
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(4) a protecting group selected from an alcohol-derived protecting group, an amide-derived protecting group, a carbonyl-derived protecting group, a carboxyl-derived protecting group, a phosphate-derived protecting group, and an alkyne-derived protecting group. The protecting group is an alcohol-derived protecting group, an amine-derived protecting group, a carbonyl-derived protecting group, a carboxyl-derived protecting group, a phosphate-derived protecting group, or an alkyne-derived protecting group. In addition, unless defined otherwise, the protecting group may be a moiety enabling introduction into a specific reactive group and removal thereof, in addition to the protecting groups exemplified above.

Here, R₉, R₁₀, R₁₅ and Rib are not electron-withdrawing groups.

Here, an electron-withdrawing group is a moiety having a tendency to withdraw electrons due to an inductive effect or resonance effect, and may also be referred to as a deactivating group. Examples of the electron-withdrawing groups of the above-described moieties include trifluoromethylsulfonyl (—SO₂CF₃), substituted or unsubstituted ammonium (—NR₃⁺), nitro, sulfonic acid (—SO₃H), sulfonyl (—SO₂R), nitrile, trihalomethyls (—CF₃, —CCl₃, —CBr₃, or —CI₃), haloformyls (—COCl, —COBr, or —COI), formyl (—CHO), acyl (—COR), carboxyl (—CO₂H), substituted carbonyl (—CO₂R), substituted or unsubstituted aminocarbonyl (—CONR₂), and nitroso (—N═O). In addition, unless defined otherwise, the electron-withdrawing group may further include a moiety having a tendency to withdraw electrons, in addition to the moieties exemplified above.

Accordingly, a moiety having a tendency to withdraw electrons among the moieties exemplified above or a moiety having a tendency to withdraw electrons other than the moieties exemplified above is preferably not located at R₉, R₁₀, R₁₅ or R₁₆.

In addition, at least one of R₁ to R₂₀ is preferably (1) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety, (2) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or (3) a protecting group selected from an alcohol-derived protecting group, an amine-derived protecting group, a carbonyl-derived protecting group, a carboxyl-derived protecting group, a phosphate-derived protecting group, and an alkyne-derived protecting group.

Here, as a reactive group, (a) a carboxyl group and a derivative thereof: N-hydroxysuccinimide ester, N-hydroxy benzotriazole ester, acyl halide, acyl imidazole, thioester, p-nitrophenyl ester, alkyl ester, alkenyl ester, alkynyl ester and aromatic ester; (b) hydroxyl that can be converted into ester, ether, or aldehyde; (c) haloalkyl that can be covalently attached to a different moiety by substituting a halogen, for example, with a nucleophilic moiety such as amine, a carboxylate anion, a thiol anion, a carboanion or an alkoxide ion; (d) a dienophile that can have a Diels-Elder reaction with, for example, a maleimido group; (e) an aldehyde or ketone that can form a carbonyl derivative such as an imine, a hydrazone, a semi-carbazone, or an oxime; (f) sulfonyl halide that forms a sulfoamide by having a reaction with an amine; (g) thiol that can be converted into a disulfide or have a reaction with an acyl halide; (h) amine or sulfhydryl that can be acylated, alkylated or oxidated; (i) alkene which is involved in cycloaddition, acylation, or a Michael reaction; (j) epoxide which is able to react with an amine or hydroxyl compound; (k) phosphoramidite, and other standard functional groups useful for a nucleic acid reaction may be used. Such a reactive group may be suitably selected to participate in or not to interfere with a reaction necessary for synthesizing a reactive quencher.

In another embodiment, these reactive groups may be protected with a protecting group so as not to participate in a random reaction in the presence of the protecting group.

The protecting group may be introduced by chemically converting a reactive group to impart reaction selectivity of at least some reactive groups during a continuous chemical or biological reaction process.

As the protecting group, an alcohol-derived protecting group, an amine-derived protecting group, a carbonyl-derived protecting group, a carboxylic acid-derived protecting group, a phosphate-derived protecting group, or an alkyne-derived protecting group is used. In addition, as the protecting group, unless defined otherwise, a moiety that enables introduction to a specific reactive group and removal thereof, in addition to the protecting groups exemplified above, may be used.

For example, when a reactive group is hydroxyl, as a protecting group, acetyl, benzoyl, benzyl, β-methoxyethoxymethyl, ether, diemthoxytrityl, methoxyemthyl ether, methoxytrityl, p-methoxybenzyl ether, p-methoxyphenyl ether, methylthiomethyl ether, silyl ether, trityl or a derivative thereof may be used.

In addition, when a reactive group is amino, as a protecting group, tert-butyl carbamate, benzyl carbamate, acetamide, phthalimide, p-toluene sulfonamide or a derivative thereof may be used.

For preferable examples of protecting groups, reference may be made to the content of the following references (Greene et al., PROTECTIVE GROUPS IN ORGANIC SYNTHESIS, John Wiley & Sons, New York, 1999; https://en.wikipedia.org/wiki/Protecting_group).

Quenchers according to various embodiments of the present invention are capable of binding to and labeling a target biomolecule (e.g., nucleic acid) through the above-described reactive group.

The above-described reactive groups are functional groups that are able to react with a functional group such as an amino group, an imino group, a thiol group or a hydroxyl group of a target biomolecule, and may form a covalent bond, such as an amide bond, an imide bond, a urethane bond, an ester bond, a phosphite bond, a phosphate bond, or a guanine bond, between a quencher and a target biomolecule.

On the other hand, in the quenchers represented by Formula 1, 2, 5, or 6, the case in which all of R₉ to R₁₄ or R₁₅ to R₁₉ are hydrogen is excluded.

More specifically, in the quenchers represented by Formula 1, 2, 5 or 6, when Q is represented by Formula 3, at least one of R₉ to R₁₄ is (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide,
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(3) a reactive group enabling a covalent bond with a moiety selected from carboxyl, carboxyl derivative, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(4) a protecting group selected from an alcohol-derived protecting group, an amide-derived protecting group, a carbonyl-derived protecting group, a carboxyl acid-derived protecting group, a phosphate-derived protecting group, and an alkyne-derived protecting group.

In addition, in the quenchers represented by Formula 1, 2, 5 or 6, when Q is represented by Formula 4, at least one of R₁₅ to R₁₉ may be any moiety listed in (1) to (4) described above.

While R₉ to R₁₄ or R₁₅ to R₁₉ may each be independently present as the moieties defined above, in some embodiments, at least one of R₉ to R₁₀ or R₁₅ to R₁₉ may be bound with an adjacent substituent, thereby forming a substituted or unsubstituted ring (e.g., a four-element ring, a five-element ring, a six-element ring, a ring consisting of more than 6 atoms, or a fused ring in which multiple rings are joined). In addition, the ring may be an aliphatic or aromatic ring. In addition, the ring may be a ring substituted with any moiety listed in (1) to (4) described above.

In addition, R₁₁ to R₁₀ or R₁₇ to R₂₀ may each be independently present as the moieties described above, and in some embodiments, R₁₁ to R₁₀ or R₁₇ to R₂₀ may be bound with an adjacent substituent, thereby forming a substituted or unsubstituted ring (e.g., a four-element ring, a five-element ring, a six-element ring, a ring consisting of more than 6 atoms, or a fused ring in which multiple rings are joined). In addition, the ring may be an aliphatic or aromatic ring. In addition, the ring may be a ring substituted with any moiety listed in (1) to (4) described above.

m and n may each be independently an integer of 0 to 3, and thus R₅ or R₆ may each be independently 0 to 3.

X is O, S, CR₇R₈ or SiR₇R₈, Y is O or S, R₇ and R₈ may each be independently selected from substituted or unsubstituted C₁-C₄₀ alkyl, substituted or unsubstituted C₁-C₄₀ heteroalkyl containing at least one hetero atom, substituted or unsubstituted C₃-C₃₀ cycloalkyl, substituted or unsubstituted C₃-C₃₀ heterocycloalkyl containing at least one hetero atom, substituted or unsubstituted aryls and substituted or unsubstituted heteroaryl, or bound with each other to form a substituted or unsubstituted ring (e.g., a four-element ring, a five-element ring, a six-element ring, a ring consisting of more than 6 atoms, or a fused ring in which multiple rings are joined). In addition, the ring may be an aliphatic or aromatic ring. In addition, the ring may be a ring substituted with any moiety listed in (1) to (4) described above.

In addition, R₁ and R₂ may be bound with each other to form a substituted or unsubstituted ring, or R₁ and/or R₂ may be bound with adjacent R₅ to form a substituted or unsubstituted ring.

In addition, R₃ and R₄ may be bound with each other to form a substituted or unsubstituted ring, or R₃ and/or R₄ may be bound with adjacent R₆ to form a substituted or unsubstituted ring.

When adjacent moieties are bound with each other to form a substituted ring, any carbon in the ring may be substituted with any moiety listed in (1) to (4) described above.

In addition, when any moiety of R₁ to R₂₀ is a substitution moiety, any carbon in the moiety may be substituted with any moiety listed in (1) to (4) described above.

In the present invention, when Ra (a is a natural number between 1 to 20) is alkenyl or alkynyl, sp²-hybrid carbon of alkenyl or sp-hybrid carbon of alkenyl may be directly bound, or indirectly bound by spa-hybrid carbon of alkyl binding to sp²-hybrid carbon of alkenyl or sp-hybrid carbon of alkynyl.

In the present invention, a Cₐ-C_{b} moiety is a moiety that has a to b carbon atoms. For example, Cₐ-C_{b} alkyl is a saturated aliphatic group, including linear and branched alkyls having a to b carbon atoms. The linear or branched alkyls may have 40 or less Cₐ-C_{b} functional groups (e.g., C₁-C₁₀ linear or C₃-C₁₀ branched) in the main chain thereof.

Specifically, the alkyl may be methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, or n-octyl.

In addition, in the present invention, alkoxy is either of an —O-(alkyl) group and an —O-(unsubstituted cycloalkyl) group, and is linear or branched hydrocarbon having one or more ether groups and 1 to 10 carbon atoms.

Specific examples of the alkoxy include methoxy, ethoxy, propoxy, isopropoxy, n-butoxy, tert-butoxy, sec-butoxy, n-pentoxy, n-hexoxy, 1,2-dimethylbutoxy, cyclopropyloxy, cyclobutyloxy, cyclopentyloxy, and cyclohexyloxy, but the present invention is not limited thereto.

In addition, in the present invention, halogen is fluoro (—F), chloro (—Cl), bromo (—Br), or iodo (—I), and haloalkyl is alkyl substituted with the above-described halogen. For example, halomethyl is methyl in which at least one of the hydrogens is replaced with halogen (—CH₂X, —CHX₂ or —CX₃).

In the present invention, “aralkyl” is the generic term for —(CH₂)ₙAr, which is a moiety in which aryl substitutes for a carbon of alkyl. Examples of the aralkyl include benzyl (—CH₂C₆H₅) and phenethyl (—CH₂CH₂C₆H₅).

In the present invention, aryl is, unless defined otherwise, an unsaturated aromatic ring including a single ring, or multiple rings (preferably, 1 to 4 rings) conjugated or connected by a covalent bond. Non-limiting examples of the aryl include phenyl, biphenyl, o-terphenyl, m-terphenyl, p-terphenyl, 1-naphthyl, 2-naphthyl, 1-anthryl, 2-anthryl, 9-anthryl, 1-phenanthrenyl, 2-phenanthrenyl, 3-phenanthrenyl, 4-phenanthrenyl, 9-phenanthrenyl, 1-pyrenyl, 2-pyrenyl, and 4-pyrenyl.

In the present invention, heteroaryl is a moiety in which one or more carbon atoms in the aryl defined above are substituted with a non-carbon atom such as nitrogen, oxygen or sulfur. Non-limiting examples of the heteroaryl include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, quinolinyl, isoquinolinyl, cinnolinyl, phthalazinyl, quinazolinyl, quinoxalinyl, pteridinyl, quinuclidinyl, carbazoyl, acridinyl, phenazinyl, phenothizinyl, phenoxazinyl, purinyl, benzimidazolyl, benzothiazolyl, and analogs conjugated therewith.

In the present invention, unless defined otherwise, a hydrocarbon ring (cycloalkyl) or a hydrocarbon ring having a hetero atom (heterocycloalkyl) may be understood as a cyclic structure of an alkyl or heteroalkyl, respectively.

Non-limiting examples of the cycloalkyls include cyclopentyl, cyclohexyl, 1-cyclohexenyl, 3-cyclohexenyl, and cycloheptyl. Non-limiting examples of the heterocycloalkyl s include 1-(1,2,5,6-tetrahydropyrinyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothiene-2-yl, tetrahydrothiene-3-yl, 1-piperazinyl, and 2-piperazinyl.

In addition, the cycloalkyl or heterocycloalkyl may have a form in which cycloalkyl, heterocycloalkyl, aryl or heteroaryl is conjugated or connected by a covalent bond.

Here, the polyalkyleneoxide is a water-soluble polymer moiety, and examples of such polyalkyleneoxides include polyethylene glycol (PEG), polypropylene glycol (PPG), a polyethylene glycol-polypropylene glycol (PEG-PPG) copolymer, and N-substituted methacrylamide-containing polymer and copolymer.

The polyalkyleneoxide may be additionally substituted, as needed, as long as the characteristics of the polymer are maintained. For example, the substitution may be a chemical bond for increasing or decreasing the chemical or biological stability of a polymer. As a specific example, any carbon or terminal carbon in the polyalkyleneoxide may be substituted with hydroxy, alkyl ether (methyl ether, ethyl ether, propyl ether or the like), carboxylemethyl ether, carboxyethyl ether, benzyl ether, dibenzylmethyl ether, or dimethylamine. In one embodiment, the polyalkyleneoxide may be polyethyleneoxide terminated with methyl ether (mPEG), wherein mPEG is represented by the formula —(CH₂CH₂O)ₙCH₃, whose size may change depending on the size of n corresponding to the number of ethylene glycol repeat units.

In addition, the quenchers represented by Formulas 1, 2, 5 and 6 may have a structure further including a counter ion. The counter ion, which is an organic or inorganic anion, may be suitably selected in consideration of the solubility and stability of the quenchers.

Examples of the counter ions of the quencher according to one embodiment of the present invention include inorganic anions such as a phosphoric acid hexafluoride ion, a halogen ion, a phosphoric add ion, a perchloric acid ion, a periodic acid ion, an antimony hexafluoride ion, a tartaric acid hexafluoride ion, a fluoroboric acid ion, and a tetrafluoride ion; and organic anions such as a thiocyanate ion, a benzen_esulfonic acid ion, a napInhalenesulfonic acid ion, a p-toluenesulfonic acid ion, an alkyl sulforric add ion, a benzenecarboxyhe acid ion, an alkylcarboxylic acid ion, a trihaloalkylcarboxylic acid ion, an alkyl sulfonic acid ion, a trihaloalkylsuifonic acid ion, and a nicotinic acid ion. In addition, metal compound ions such as bisphenylditol, thiobisphenol chelate, and bisdiol-α-diketone, metal ions such as sodium and potassium, and quaternary ammonium salts may also be selected as the counter ions.

Examples of the quenchers represented by Formulas 1, 2, 5 and 6 are as follows. Compounds 1 to 42 exemplified below may be synthesized by a known synthesis method with reference to preparation examples disclosed herein or the content defined herein relating to the quenchers represented by Formulas 1, 2, 5 and 6.

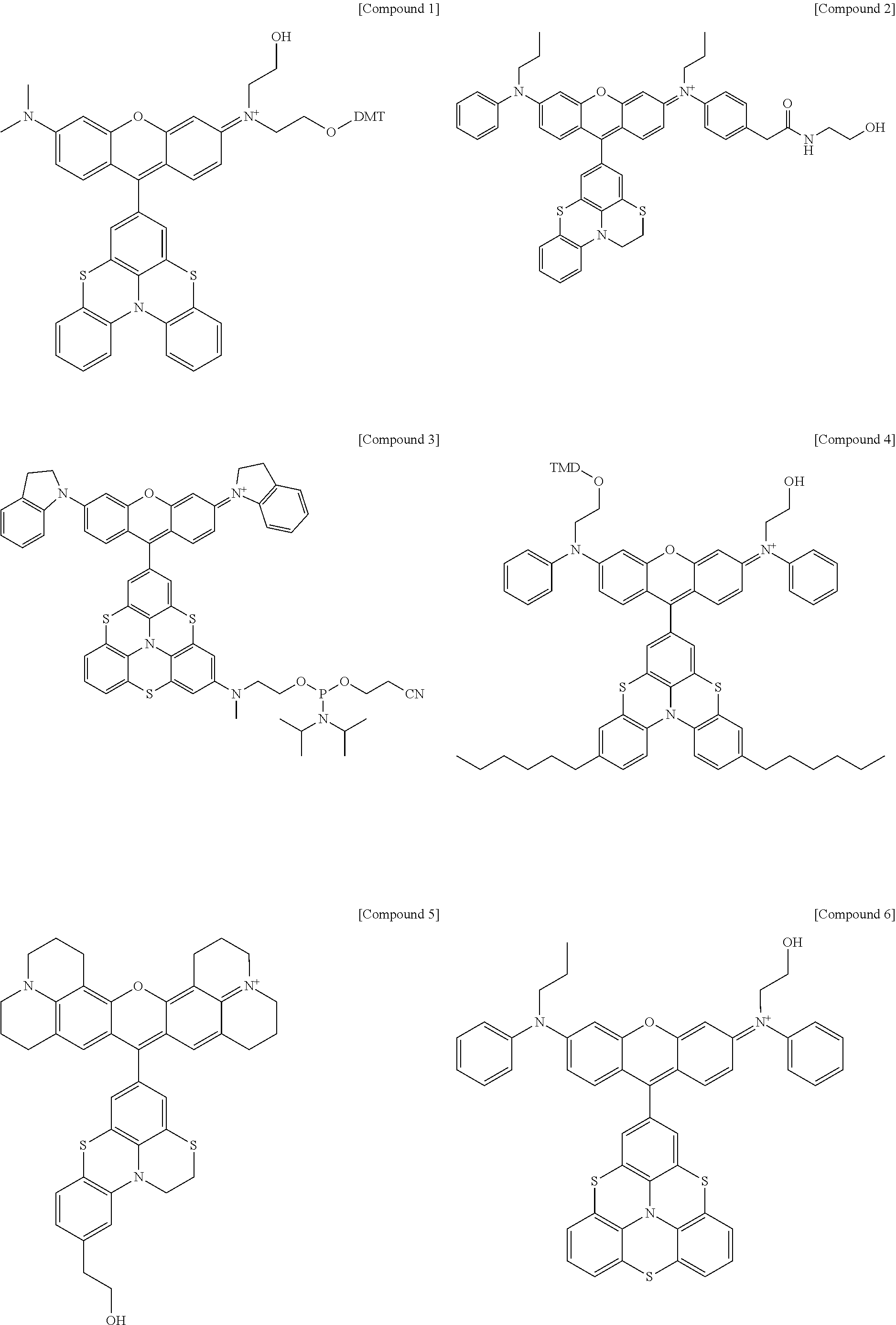

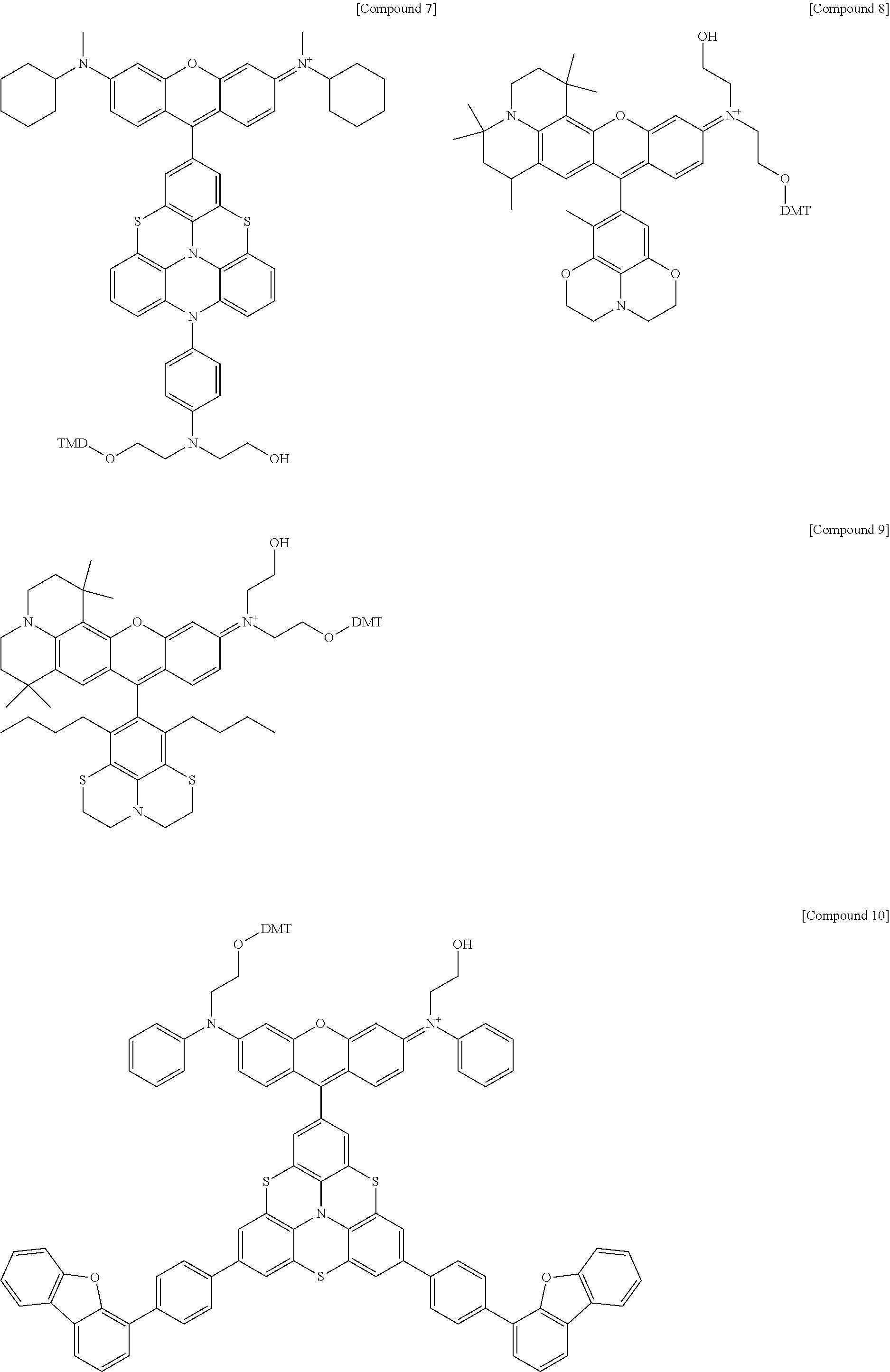

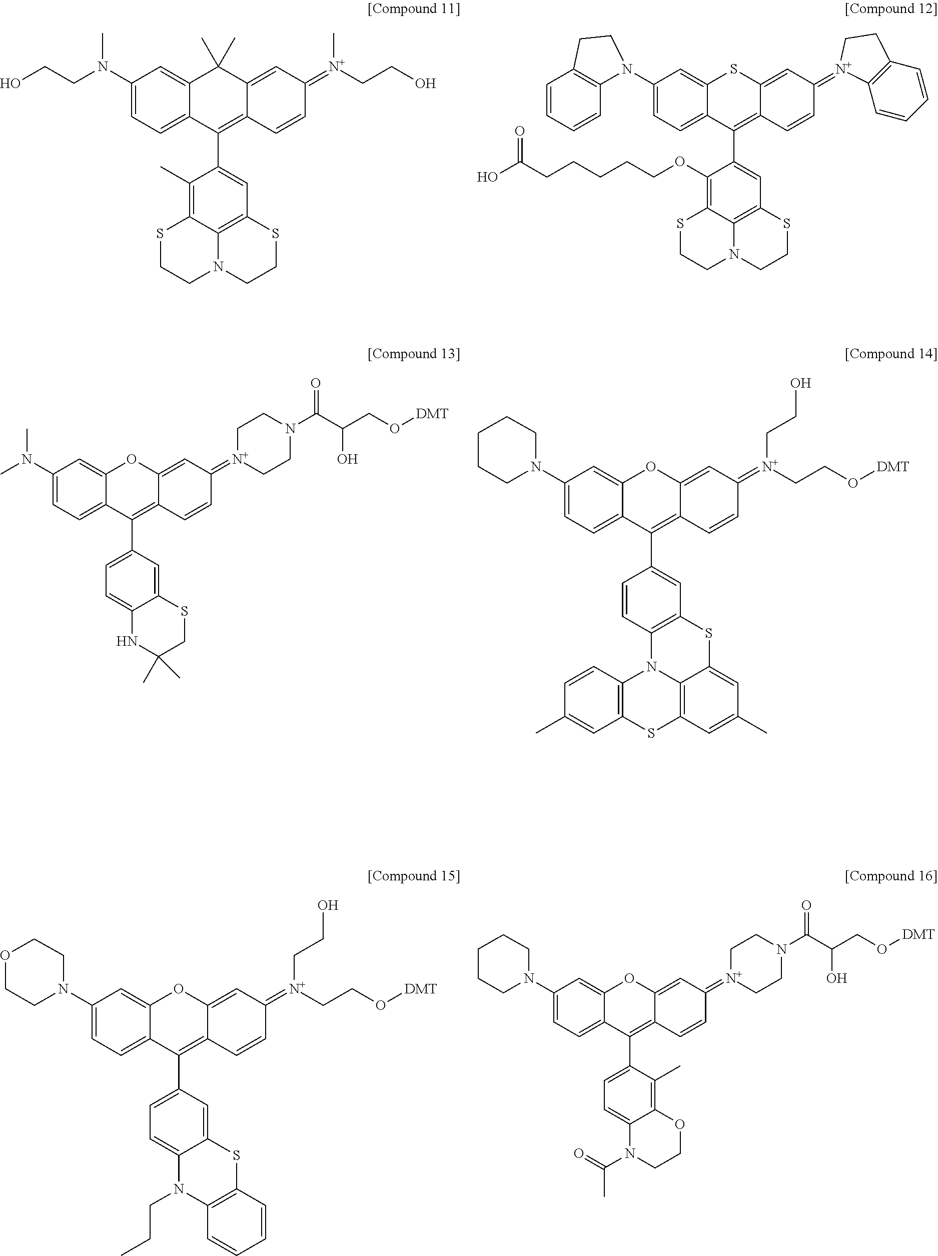

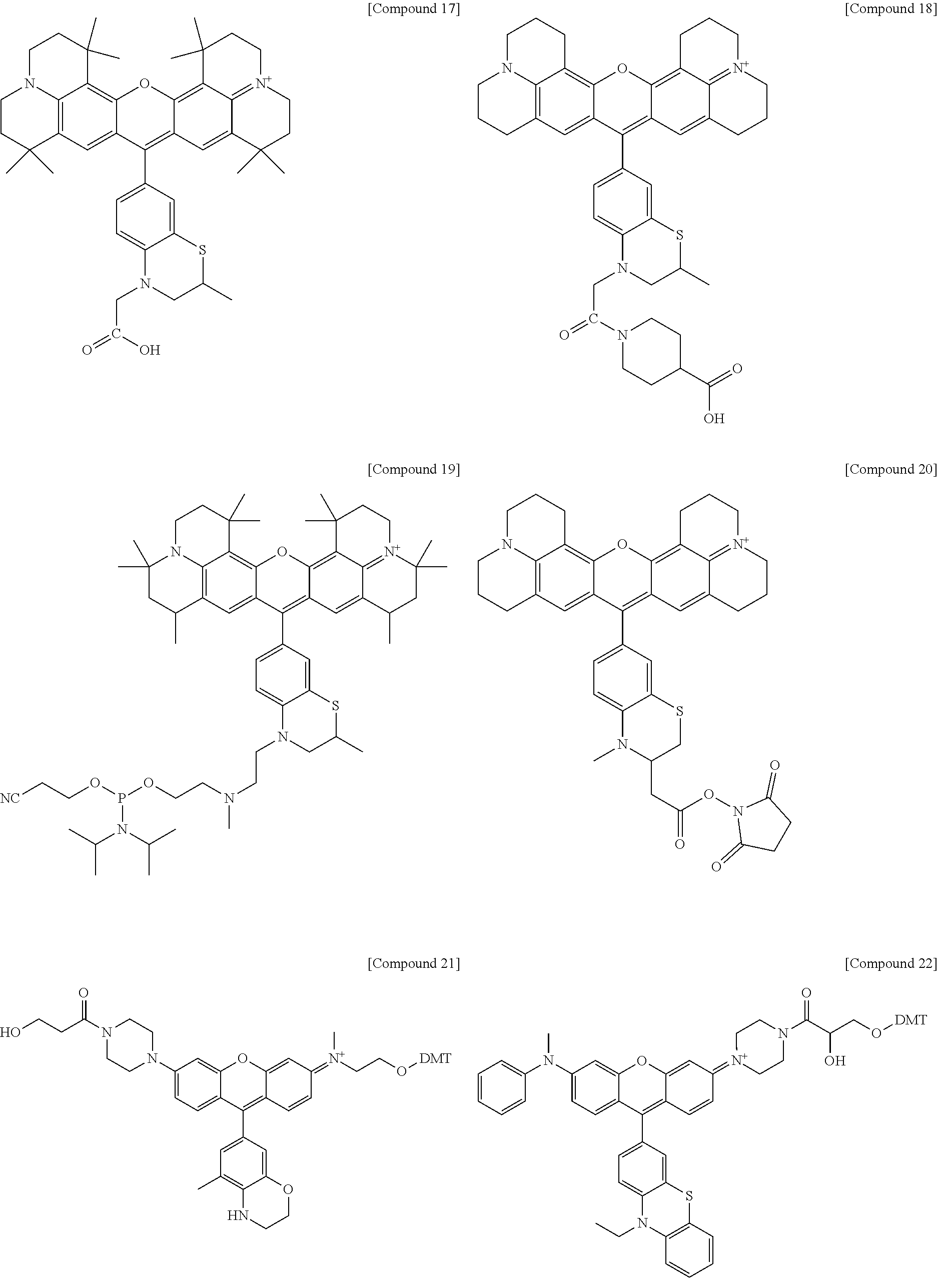

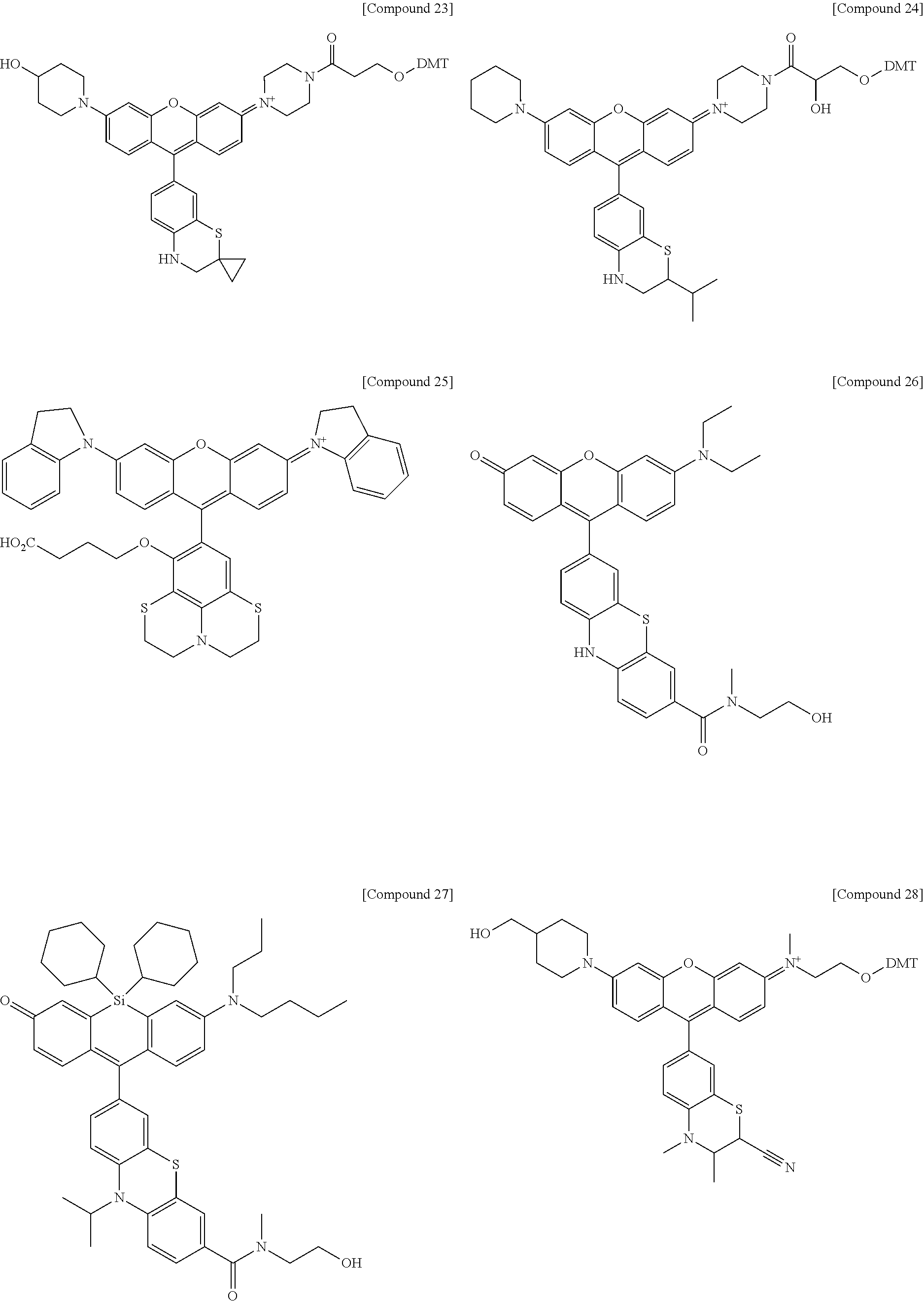

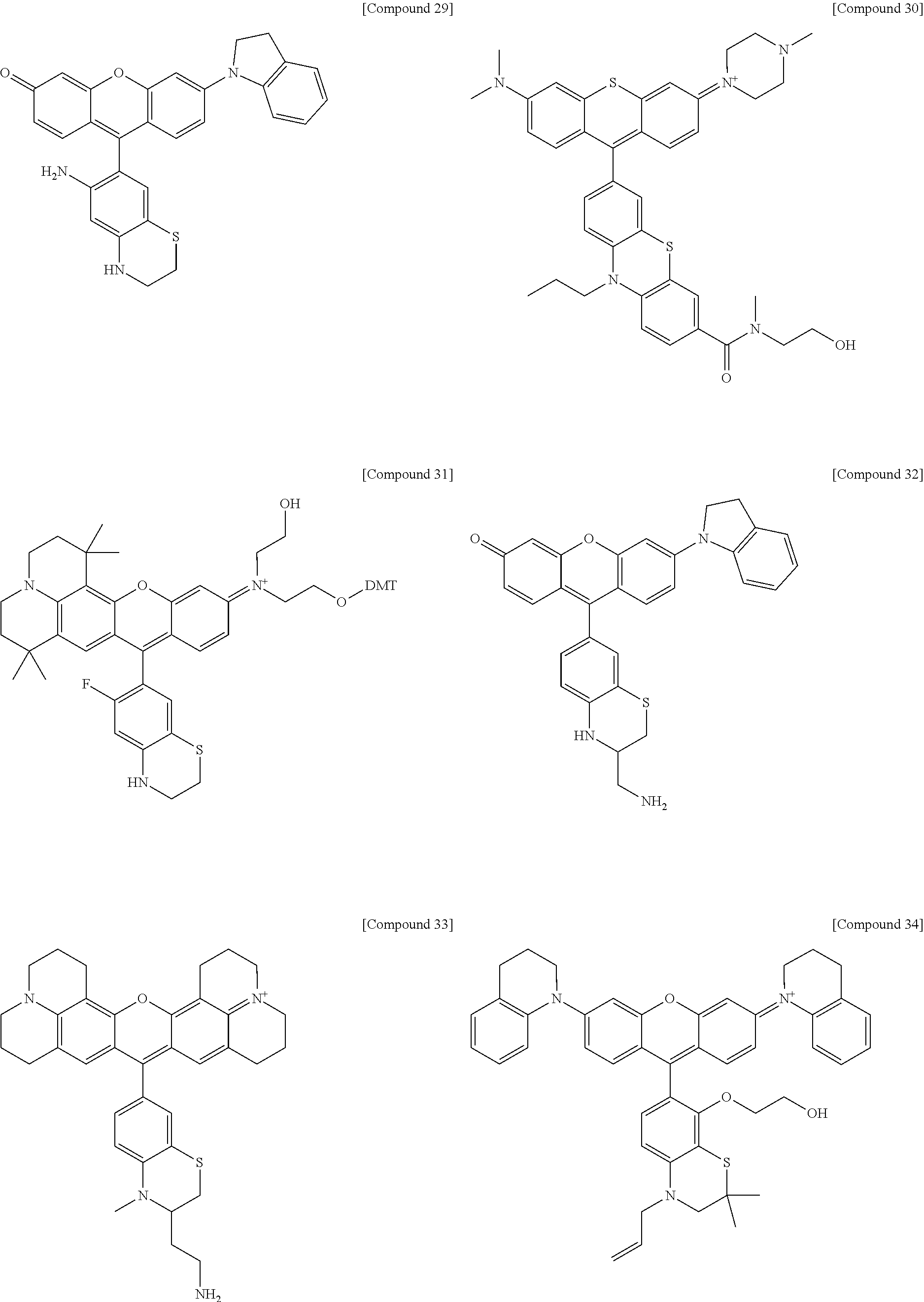

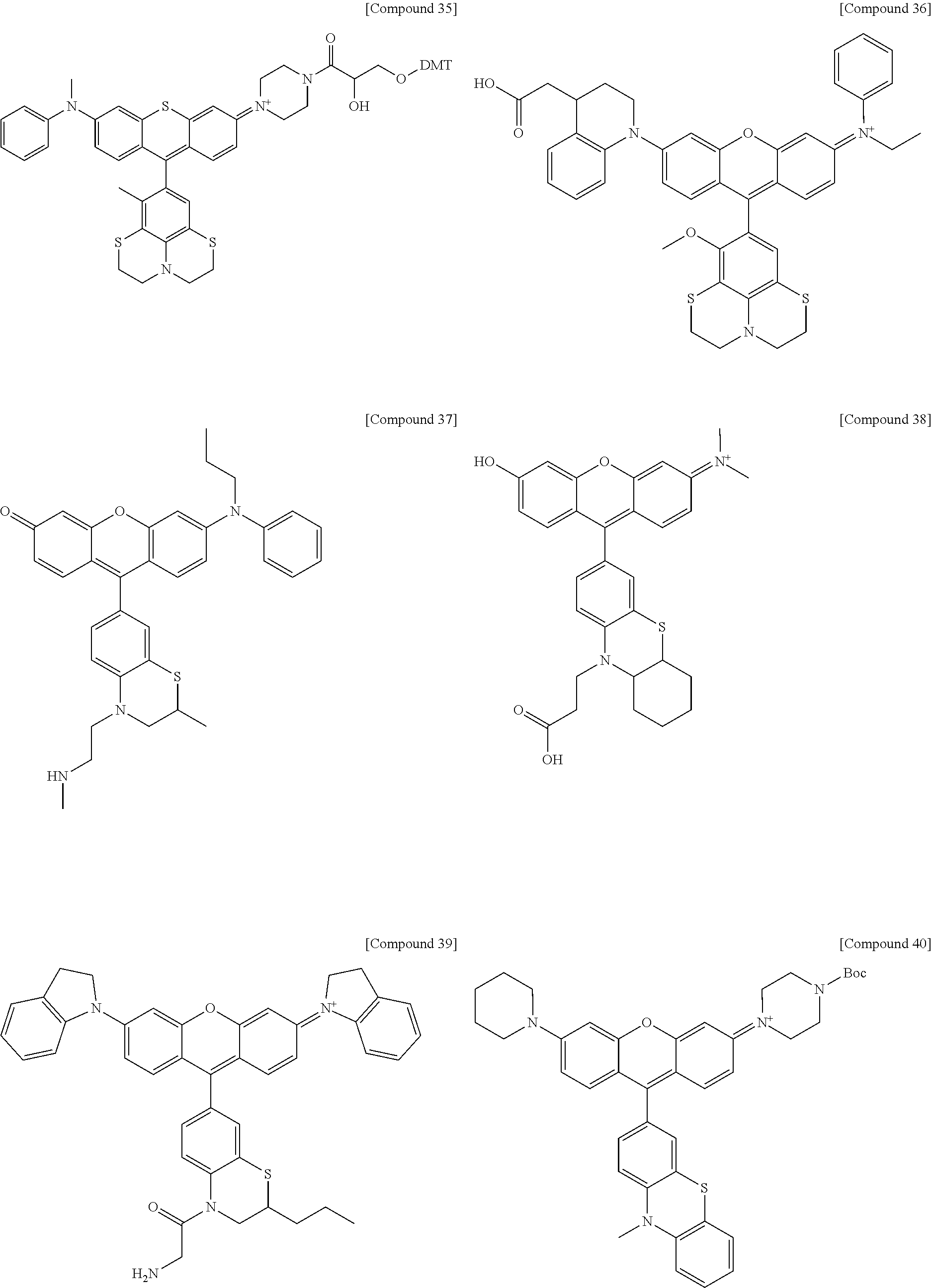

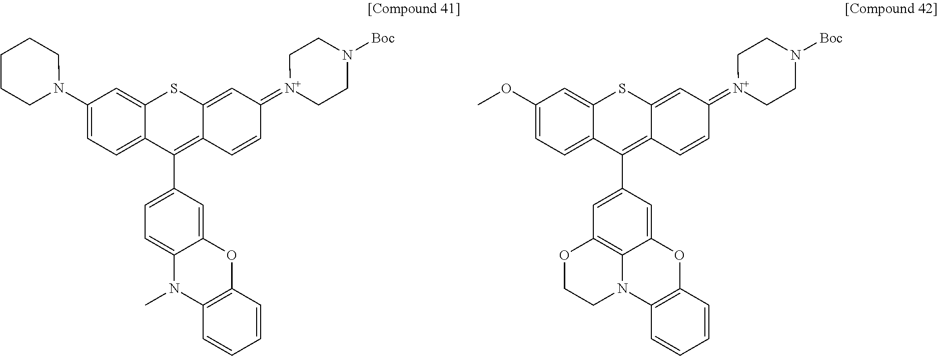 [SMILES: CN1c2ccccc2Oc2cc(-c3c4ccc(=[N+]5CCN(C(=O)OC(C)(C)C)CC5)cc-4sc4cc(N5CCCCC5)ccc34)ccc21 ; COc1ccc2c(-c3cc4c5c(c3)Oc3ccccc3N5CCO4)c3ccc(=[N+]4CCN(C(=O)OC(C)(C)C)CC4)cc-3sc2c1]

A biomolecule targeting the quenchers represented by Formulas 1, 2, 5 and 6 disclosed herein may be at least one selected from an antibody, a lipid, a protein, a peptide, a carbohydrate, and a nucleic acid (including a nucleotide).

Specific examples of the lipids include fatty acids, phospholipids, and lipopolysaccharides, and specific examples of the carbohydrates include monosaccharides, di saccharides, and polysaccharides (e.g., dextran).

Here, as any moiety of the quenchers represented by Formulas 1, 2, 5 and 6, or a moiety for reacting with a reactive group binding to the quenchers represented by Formulas 1, 2, 5 and 6, the biomolecule may include at least one selected from amino, sulphydryl, carbonyl, hydroxyl, carboxyl, phosphate and thiophosphate, or have a derivative thereof.

In addition, the biomolecule may be an oxy- or deoxy-polynucleic acid which includes at least one selected from amino, sulphydryl, carbonyl, hydroxyl, carboxyl, phosphate and thiophosphate, or a derivative thereof.

Moreover, the quencher represented by Formula 1, 2, 5 or 6 may be used to label a drug including at least one selected from amino, sulphydryl, carbonyl, hydroxyl, carboxyl, phosphate and thiophosphate, a hormone (including a receptor ligand), a receptor, an enzyme or enzyme substrate, a cell, a cell membrane, a toxin, a microorganism, or a nano biomaterial (polystyrene microsphere or the like), in addition to the biomolecule.

Oligonucleotide, Composition for Detecting Nucleic Acid and Support for Detecting Nucleic Acid, Including Novel Quencher

According to another aspect of the present invention, an oligonucleotide including at least one selected from the quenchers represented by Formulas 1, 2, 5 and 6 is provided.

The oligonucleotide is a polymer of one to hundreds of nucleotides, and examples of such oligonucleotides include DNA, RNA, and PNA. In addition, such oligonucleotides include analogs thereof, for example, those with chemically modified nucleotides or those that are easily modified by those of ordinary skill in the art, such as those to which a sugar binds, and are single-stranded or double-stranded.

The oligonucleotide preferably includes a probe. Such a probe is more preferably a probe capable of complementarily binding to a targeted nucleic acid, but the present invention is not limited thereto. Here, the probe may be selected from a nucleic acid, a peptide, a saccharide, an oligonucleotide, a protein, an antibody, or a combination thereof, but the present invention is not limited thereto.

In one embodiment, the oligonucleotide may include a fluorophore. For example, the 5′ end of the oligonucleotide may be labeled with a fluorophore, and the 3′ end thereof may be labeled with at least one selected from the quenchers represented by Formulas 1, 2, 5 and 6. Between the 5′ end and the 3′ end,
a probe that is able to complimentarily bind to the targeted nucleic acid may be located.

The fluorophore may refer to the types of the fluorophores disclosed in the following references (Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995)).

In addition, non-limiting examples of the fluorophores that can be used herein include 4-acetamido-4′-isothiocyanatostilbene-2,2′disulfonic acid, acridine and derivatives thereof, 5-(2′-aminoethyl)aminonaphthalene-1-sulfonic acid (EDANS), 4-amino-N-[3-vinyl sulfonyl)phenyl]naphthalimide-3,5 di sulfonate, N-(4-anilino-1-naphthyl)maleimide, anthranilamide, BODIPY, Brilliant Yellow, coumarins (7-amino-4-methylcoumarin (AMC, Coumarin 120), and 7-amino-4-trifluoromethylcouluarin (Coumaran 151)) and derivatives thereof, cyanine dyes, cyanosine, 4′,6-diaminidino-2-phenylindole (DAPI), 5′,5″-dibromopyrogallol-sulfonaphthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansylchloride), 4-(4′-dimethylaminophenylazo)benzoic acid (DABCYL), 4-dimethylaminophenylazophenyl-4′-isothiocyanate (DABITC), eosin and a derivative thereof (eosin isocyanate), erythrosine and derivatives thereof (erythrosine B, erythrosine isocyanate), ethidium, fluorescein and derivatives thereof (5-carboxyfluorescein (FAM)), 5-(4,6-dichlorotriazin-2-yl)aminofluorescein (DTAF), 2′,7′-dimethoxy-4′5′-dichloro-6-carboxyfluorescein (JOE), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene and derivatives thereof (pyrene butyrate, succinimidyl 1-yprene butyrate), quantum dots, Reactive Red 4 (Cibacron Brilliant Red 3B-A), rhodamine and derivatives thereof (6-carboxy-X-rhodamine, 6-carboxyrhodamine, rhodamine B, rhodamine 123, rhodamine X isocyanate, sulforhodamine B, sulforhodamine 101, tetramethyl rhodamine, and tetramethyl rhodamine isocyanate), riboflavin, rosolic acid, pyrene, carbopyronin, oxazine, xanthene, thioxanthene, and terbium chelate derivatives.

In addition, the oligonucleotide according to the present invention may further include a minor groove binder (MGB) to improve the binding strength with a nucleic acid.

Such an oligonucleotide may be used in various ways in the fields of chemistry and biology. Particularly, it may be useful for real time PCR or microassay, but the present invention is not limited thereto.

In addition, according to another aspect of the present invention, a composition for detecting a nucleic acid, including the oligonucleotide, is provided.

The composition for detecting a nucleic acid according to one embodiment of the present invention may further include an enzyme, a solvent (a buffer or the like) and other reagents for a reaction with a target biomolecule, in addition to an oligonucleotide including all of the quencher represented by Formula 1, 2, 5 or 6, MGB, and the fluorophore.

Here, as the solvent, a buffer selected from the group consisting of a phosphate buffer, a carbonate buffer and a Tris buffer, an organic solvent selected from dimethyl sulfoxide, dimethylformamide, dichloromethane, methanol, ethanol and acetonitrile, or water may be used, and it is possible to adjust solubility by introducing various functional groups to the quencher according to the type of solvent.

In addition, according to still another aspect of the present invention, a support for detecting a nucleic acid, which includes the quencher represented by Formula 1, 2, 5 or 6, a support, and a linker connecting the quencher and the support, is provided.

Accordingly, a biomolecule in a sample may be fixed on a supporting matrix through interaction with the quencher adhered onto the support.

The support may be manufactured with at least one selected from glass (e.g., CPG), cellulose, nylon, acrylamide gel, dextran, polystyrene, alginate, collagen, peptides, fibrin, hyaluronic acid, agarose, polyhydroxyethylmethacrylate, polyvinyl alcohol, polyethylene glycol, polyethyleneoxide, polyethylene glycol diacrylate, gelatin, Matrigel, polylactic acid, carboxymethylcellulose, chitosan, latex, and Sepharose, and may be formed as a bead or a membrane.

Here, the linker is a part connecting the quencher and the support, and any material capable of connecting the quencher and the support may be used as a linker intended by the present invention.

For example, the linker may be selected from substituted or unsubstituted C₁-C₃₀ alkyl, substituted or unsubstituted C₃-C₃₀ cycloalkyl, substituted or unsubstituted C₂-C₃₀ heteroalkyl having at least one hetero atom, substituted or unsubstituted C₂-C₃₀ heterocycloalkyl having at least one hetero atom, substituted or unsubstituted C₂-C₃₀ alkenyl, substituted or unsubstituted C₆-C₃₀ aryl, substituted or unsubstituted C₃-C₃₀ heteroaryl, amide (—CONH—), ester (—COO—), ketone (—CO—), nucleoside, and any combination thereof.

Examples of the connection structures between the supports and the quenchers mediated by the linkers are as follows.

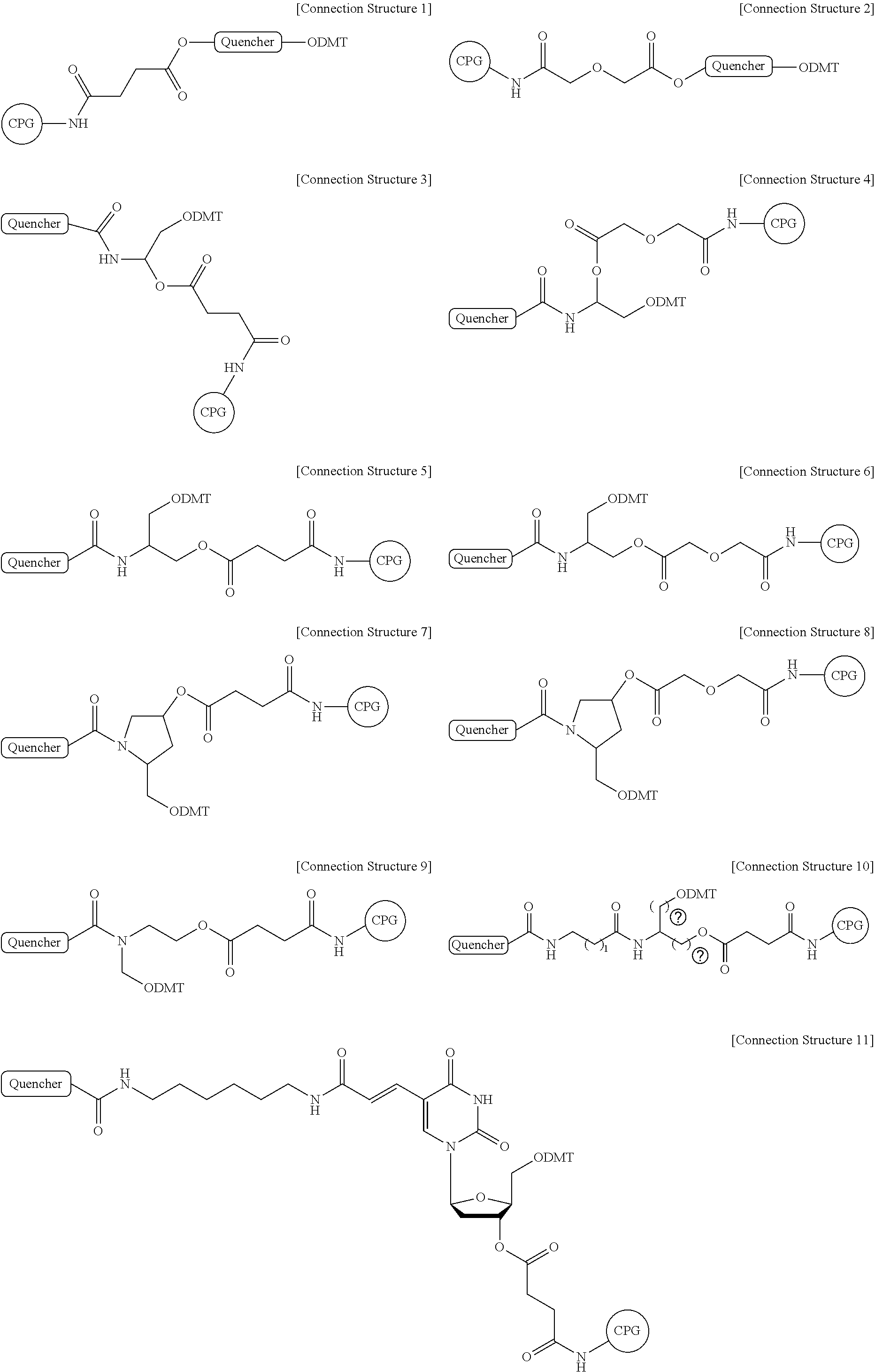

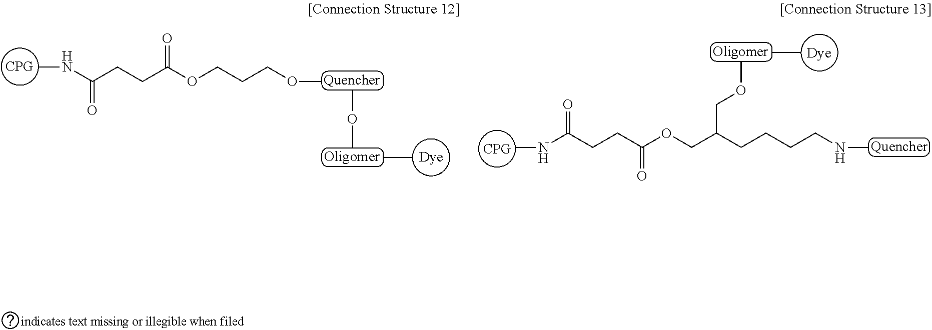

The linker merely connects a quencher and a support, but does not affect any other reaction of a quencher or fluorophore, or fluorescence or quenching actions.

Method of Detecting Nucleic Acid

According to one embodiment of the present invention, a method of labeling a target nucleic acid through reaction with a quencher-labeled probe may be implemented. In addition, a biomolecule labeling method using a target-specific interaction may be implemented by introducing an appropriate reactive group to a quencher according to the type of target biomolecule. Moreover, a method of identifying the quencher-labeled biomolecule through electrophoresis may be implemented.

DNA Microarray

A DNA microarray is for measuring the fluorescence of a target nucleic acid by preparing a single-stranded probe nucleic acid which labels a target nucleic acid through reaction with a dye and has a complementary base sequence to the target nucleic acid, and hybridizing the probe nucleic acid with the target nucleic acid denatured into a single strand on a substrate.

In the labeling method, when gene expression is investigated, as the probe nucleic acid immobilized to the substrate, cDNA, which is prepared by amplifying a cDNA library, genome library, or any of all genomes as a template through PCR, may be used.

In addition, for investigation of a gene mutation, various oligonucleotides corresponding to mutations may be synthesized based on a known sequence serving as a reference and used.

A proper method for immobilizing the probe nucleic acid on the substrate may be selected according to the type of nucleic acid or substrate. For example, a method for electrostatic bonding to a substrate surface-treated with a cation such as polylysine using the charge of DNA may also be used.

The target nucleic acid denatured into a single strand is immobilized on the substrate, and hybridized with the oligonucleotide. Here, the 5′ end of the oligonucleotide is labeled with a fluorophore, and the 3′ end thereof is labeled with at least one selected from the quenchers represented by Formulas 1, 2, 5 and 6. Between the 5′ end and the 3′ end, a probe that is able to complimentarily bind to the targeted nucleic acid may be located.

Hybridization is preferably performed at room temperature to 70° C. for approximately 2 to 48 hours. Through hybridization, the target nucleic acid having a complementary base sequence with the probe nucleic acid is selectively bound with the probe nucleic acid. Afterward, the substrate is washed and dried at room temperature.

Here, the oligonucleotide is hybridized to the target nucleic acid by the probe, but the fluorophore at the 5′ end is present in a quenched state by the quencher at the 3′ end.

Subsequently, the oligonucleotide hybridized to the target nucleic acid is elongated by a polymerase and separated from the target nucleic acid by the exonuclease activity of the polymerase and degraded. The fluorophore at the 5′ end and the quencher at the 3′ end of the oligonucleotide are separated from each other, and thus the fluorophore may exhibit fluorescence.

Here, the intensity of the generated fluorescence is measured to measure the amplification amount of the target nucleic acid.

Hereinafter, specific examples of the present invention will be presented. However, the following examples are only for exemplifying or explaining the present invention in detail, and the present invention is not limited thereto.

### Preparation Example 1. Synthesis of Compound 40

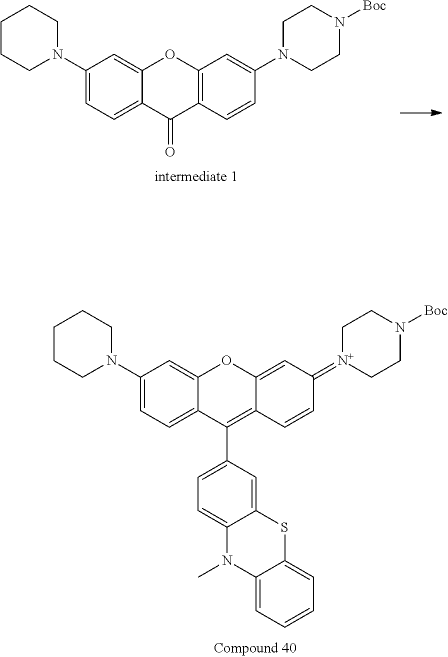 [SMILES: CC(C)(C)OC(=O)N1CCN(c2ccc3c(=O)c4ccc(N5CCCCC5)cc4oc3c2)CC1 ; CN1c2ccccc2Sc2cc(-c3c4ccc(=[N+]5CCN(C(=O)OC(C)(C)C)CC5)cc-4oc4cc(N5CCCCC5)ccc34)ccc21]

3-bromo-10-methyl-10H-phenothiazine (3.2 g, 11 mmol) and tetrahydrofuran (20 mL) were put into a 250 mL 3-neck reactor, and stirred under a nitrogen atmosphere at −78° C. for 5 minutes. 1.6M n-butyl lithium (4.5 mL, 7 mmol) was slowly added dropwise to the reactor, and then stirred at −78° C. for 1 hour. Intermediate 1 (1.7 g, 4 mmol; synthesized with reference to Korean Unexamined Patent Application Publication No. 10-2019-0062162) was dissolved in tetrahydrofuran (50 mL) and added dropwise to the reactor, followed by stirring at room temperature for 12 hours. Hydrochloric acid (4 mL) was added to the reactor, vigorously stirred for 30 minutes, concentrated, and then purified using a column, thereby synthesizing 0.9 g of a black solid (yield: 31%).

¹H-NMR of the obtained Compound 1 is as follows.

¹H-NMR (400 MHz, CDCl₃) δ 7.53-7.46 (m, 2H), 7.31-6.98 (m, 10H), 6.91 (d, J=8.4 Hz, 1H), 3.77-3.67 (m, 12H), 3.5 (s, 3H), 1.79 (bs, 6H), 1.49 (s, 9H)

### Experimental Example 1. Evaluation of Absorption Property of Quencher

To evaluate the absorption property of Compound 40 synthesized according to Preparation Example 1 as a quencher, the absorption spectrum (PBS solvent), molar extinction coefficient (ε, Lmol⁻¹ cm⁻¹) and quantum efficiency (φ) of Compound 40 were measured by known methods.

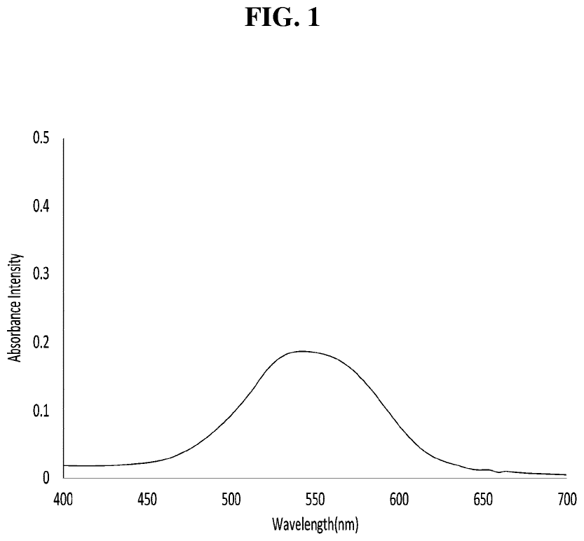

The measurement results are shown in FIG. 1 and Table 1 below.

TABLE 1

| | | | Molar | Quantum |
|---|---|---|---|---|
| | | λ_{Max} | extinction | efficiency |
| Classification | Solvent | (nm) | coefficient | (φ) |
| Compound 40 | PBS | 546 | 38,000 | 0.002 |

As shown in FIG. 1 and Table 1, it can be confirmed that the compounds according to various embodiments of the present invention can serve as quenchers which do not emit light even while absorbing great amounts of light due to a high extinction coefficient and quantum efficiency close to zero.

Although embodiments of the present invention have been described above, the spirit of the present invention is not limited thereto, and it will be understood by those of ordinary skill in the art that the present invention may be modified and altered in various ways by adding, altering, or deleting a component without departing from the spirit of the present invention defined in the appended claims, and such modifications and alterations will also be included in the scope of the present invention.

What is claimed is:

1. A quencher represented by Formula 1 or 2 below: 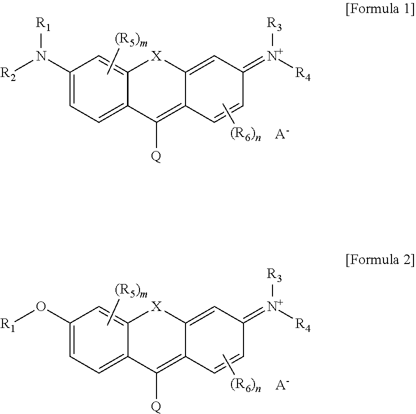
Wherein,
Q is represented by Formula 3 or 4 below, 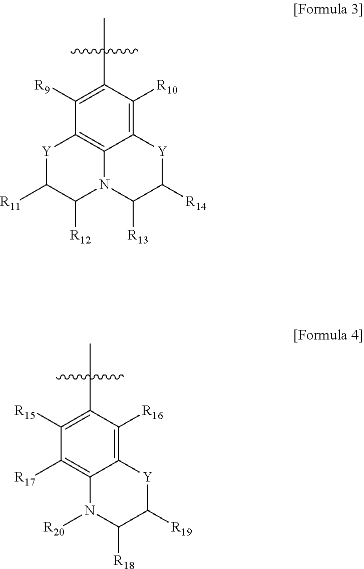
R₁ to R₆ and R₉ to R₂₀ are each independently (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide,
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(4) a protecting group selected from an alcohol-derived protecting group, an amide-derived protecting group, a carbonyl-derived protecting group, a carboxyl acid-derived protecting group, a phosphate-derived protecting group, and an alkyne-derived protecting group,
m and n are each independently an integer of 0 to 3,
X is O, S, CR₇R₈, or SiR₇R₈,
Y is O or S,
R₇ and R₈ are each independently selected from substituted or unsubstituted C₁-C₄₀ alkyl, substituted or unsubstituted C₁-C₄₀ heteroalkyl having at least one hetero atom, substituted or unsubstituted C₃-C₃₀ cycloalkyl, substituted or unsubstituted C₃-C₃₀ heterocycloalkyl having at least one hetero atom, substituted or unsubstituted aryl, and substituted or unsubstituted heteroaryl, or form a ring by bonding them together,
at least one of R₁ to R₂₀ is
(1) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety,
(2) a reactive group enabling a covalent bond with a moiety selected from carboxyl, carboxyl derivatives, hydroxyl, haloalkyls, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group, or
(3) a protecting group selected from an alcohol-derived protecting group, an amide-derived protecting group, a carbonyl-derived protecting group, a carboxylic acid-derived protecting group, a phosphate-derived protecting group, and an alkyne-derived protecting group,
R₉, R₁₀, R₁₅ and R₁₆ are not electron-withdrawing groups, and
the case in which all of R₉ to R₁₄ or R₁₅ to R₁₉ are hydrogen is excluded.

2. The quencher of claim 1, wherein the quencher exists as at least one structure selected from Formula 1 and Formula 5, which is a resonance structure of Formula 1:

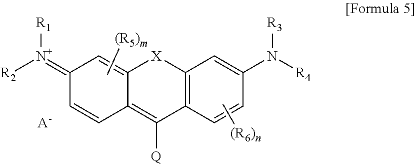

3. The quencher of claim 1, wherein the quencher exists as at least one structure selected from Formula 2 and Formula 6, which is a resonance structure of Formula 2:

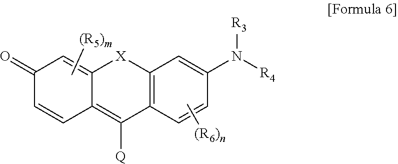

4. The quencher of claim 1, wherein R₁ and R₂ are bound with each other to form a substituted or unsubstituted ring.

5. The quencher of claim 1, wherein at least one selected from R 1 and R₂ is bound with adjacent R₅ to form a substituted or unsubstituted ring.

6. The quencher of claim 1, wherein R₃ and R₄ are bound with each other to form a substituted or unsubstituted ring.

7. The quencher of claim 4, wherein the ring is substituted with at least one selected from (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-050 aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide;
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety;
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group; and
(4) a protecting group selected from an alcohol-derived protecting group, an amide-derived protecting group, a carbonyl-derived protecting group, a carboxyl-derived protecting group, a phosphate-derived protecting group, and an alkyne-derived protecting group.

8. The quencher of claim 1, wherein at least one selected from R₃ and R₄ is bound with adjacent R₆ to form a substituted or unsubstituted ring.

9. The quencher of claim 1, wherein the reactive group is protected with a protecting group.

10. The quencher of claim 1, wherein at least one of R₉ to R₁₀ or R₁₅ to R₁₉ is bound with an adjacent substituent to form a substituted or unsubstituted ring.

11. The quencher of claim 10, wherein the ring is substituted with at least one selected from (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide;
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety;
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group; and
(4) a protecting group selected from an alcohol-derived protecting group, an amide-derived protecting group, a carbonyl-derived protecting group, a carboxyl-derived protecting group, a phosphate-derived protecting group, and an alkyne-derived protecting group.

12. The quencher of claim 1, wherein adjacent substituents of Ru to R₁₀ or R₁₇ to R₂₀ are bound with each other to form a ring.

13. The quencher of claim 12, wherein the ring is substituted with at least one selected from (1) a moiety selected from hydrogen, deuterium, substituted or unsubstituted C₁-C₄₀ alkyl, substituted or unsubstituted C₁-C₄₀ heteroalkyl having at least one hetero atom, substituted or unsubstituted C₂-C₄₀ alkenyl, substituted or unsubstituted C₂-C₄₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O⁻), substituted or unsubstituted C₁-C₄₀ alkoxy, substituted or unsubstituted C₃-C₄₀ cycloalkyloxy, substituted or unsubstituted C₅-C₄₀ aryloxy, substituted or unsubstituted C₂-C₄₀ heteroaryloxy, substituted or unsubstituted C₅-C₅₀ aryl, substituted or unsubstituted C₂-C₅₀ heteroaryl, substituted or unsubstituted C₅-C₅₀ aralkyl, substituted or unsubstituted C₁-C₄₀ alkylthio, substituted or unsubstituted C₅-C₄₀ arylthio, substituted or unsubstituted C₃-C₄₀ cycloalkylthio, substituted or unsubstituted C₂-C₄₀ heteroarylthio, substituted or unsubstituted acylamino, acyloxy, substituted or unsubstituted phosphino, carboxylate (—CO₂⁻), trifluoromethylsulfonyl (—SO₂CF₃), substituted or unsubstituted ammonium, nitro, sulfonic acid (—SO₃H), sulfonate, substituted sulfonyl, substituted sulfonic ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkylene oxide;
(2) a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the moiety;
(3) a reactive group enabling a covalent bond with a moiety selected from carboxyl, carboxyl derivatives, hydroxyl, haloalkyl, dienophile, aldehyde, substituted ketone, sulfonyl halide, thiol, substituted or unsubstituted amino, sulfhydryl, alkene, alkyne, halogen, hydrazine, azido, imido, ketene, isocyanate, epoxide, and phosphoramidite, or any moiety substituted with the reactive group; and
(4) a protecting group selected from an alcohol-derived protecting group, an amide-derived protecting group, a carbonyl-derived protecting group, a carboxyl-derived protecting group, a phosphate-derived protecting group, and an alkyne-derived protecting group.

14. An oligonucleotide comprising:
the quencher of claim 1;
a minor groove binder (MGB); and
a fluorophore.

15. The oligonucleotide of claim 14, wherein the fluorophore is at least one selected from coumarin, cyanine, BODIPY, fluorescein, rhodamine, pyrene, carbopyronine, oxazine, xanthene, thioxanthene, acridine, and a derivative thereof.

16. A composition for detecting a nucleic acid, comprising the oligonucleotide of claim 14.

17. A support for detecting a nucleic acid, comprising:
the quencher of claim 1;
a support; and
a linker connecting the quencher and the support.

18. The support of claim 17, which is glass, cellulose, nylon, acrylamide gel, dextran, polystyrene, or resin.

19. The support of claim 17, wherein the linker is selected from substituted or unsubstituted C₁-C₃₀ alkyl, substituted or unsubstituted C₃-C₃₀ cycloalkyl, substituted or unsubstituted C₂-C₃₀ heteroalkyl having at least one hetero atom, substituted or unsubstituted C₂-C₃₀ heterocycloalkyl having at least one hetero atom, substituted or unsubstituted C₂-C₃₀ alkenyl, substituted or unsubstituted C₆-C₃₀ aryl, substituted or unsubstituted C₃-C₃₀heteroaryl, amide (—CONH—), ester (—COO—), ketone (—CO—), nucleoside, and any combination thereof.

20. A method of detecting a nucleic acid, comprising:
(a) preparing a reaction mixture including a target nucleic acid, a reagent necessary for amplifying the target nucleic acid, and the oligonucleotide of claim 14;
(b) amplifying the target nucleic acid in the reaction mixture by a chain polymerase reaction; and
(c) measuring the fluorescence intensity of the reaction mixture.

21. The method of claim 20, wherein Step (b) comprises:
(b-1) elongating an oligonucleotide hybridized to the target nucleic acid by a polymerase;
(b-2) separating the quencher and the fluorophore of the oligonucleotide from the target nucleic acid by the exonuclease activity of the polymerase; and
(b-3) emitting fluorescence from the fluorophore separated from the quencher.

22. The method of claim 20, further comprising:
(d) measuring the amplification amount of the target nucleic acid from the intensity of fluorescence measured in Step (c).
